(19) United States
(12) Patent Application Publication
(10) Pub. No.: US 2022/0375542 A1
(43) Pub. Date: Nov. 24, 2022

(54) SYSTEMS AND METHODS FOR GENERATING AND ANALYZING A CUSTOMIZED GENOMIC SEQUENCE FOR THERAPEUTIC APPLICATIONS

(71) Applicant: Arizona Board of Regents on Behalf of the University of Arizona, Tucson, AZ (US)

(72) Inventor: Adam David Grant, Tucson, AZ (US)

(21) Appl. No.: 17/747,796

(22) Filed: May 18, 2022

Related U.S. Application Data
(60) Provisional application No. 63/190,202, filed on May 18, 2021.

(51) Int. Cl. G16B 20/50; G16B 30/00

(52) U.S. Cl. CPC G16B 20/50; G16B 30/00

(57) ABSTRACT

Systems and methods are described for genetic analysis. In certain embodiments, the system reads a plurality of input parameters, where the input parameters include a file-path to a mutation input file and storing the mutation input file. The mutation input file is comprised of a mutated genetic sequence, and the sorting mutations in the mutation input file are based on starting position. The computer then receives data identifying chromosome location, start position, reference allele and mutated allele for each mutation within the mutation input file and loads a standardized reference genome. In certain embodiments, the GTF file identifies the location of mutated genes in the input standardized reference genome. The system then compares the mutation input file to the standardized reference genome and generates a mutation index file. The mutation index file identifies mutated nucleotides in the customized reference genome and can be used to quantify allelic expression to diagnose a genetic condition like cancer and improve therapeutic options for cancer patients.

### CROSS REFERENCE TO RELATED APPLICATION

This application claims the benefit of U.S. Provisional Application No. 63/190,202, filed May 18, 2021, the contents of which are hereby incorporated by reference herein.

### STATEMENT REGARDING FEDERALLY SPONSORED RESEARCH

This invention was made with government support under Grant No. CA211878 awarded by the National Institutes of Health. The government has certain rights in the invention.

### FIELD OF THE INVENTION

The present invention relates to computer-implemented systems and methods for generating and analyzing customized genomic sequences that are used for therapeutic applications.

### BACKGROUND OF THE INVENTION

Diseases like cancer can find their origins in mutations in the genetic sequences of cells. Sequencing is the process of determining the nucleic acid sequence—the order of nucleotides in the DNA or RNA of cells. It includes any method or technology that is used to determine the order of the four bases: adenine, guanine, cytosine, and thymine/uracil. As cancer is a genetic disease driven by heritable or somatic mutations, new DNA sequencing technologies will have a significant impact on the detection, management and treatment of disease.

DNA sequencing for use in cancer diagnostics may be computer-assisted. For example, next-generation sequencing may be used to catalogue mutations in multiple cancer types are that can be translated to diagnostic, prognostic and therapeutic targets. However, as the technology is in its infancy, there is a need in the art for a system that has the capability to increase alignment sensitivity of predefined mutations, which can be used to improve detection of circulating cancer cells, improve patient prognosis, and enhance personalized therapeutic approaches for cancer patients.

### SUMMARY OF THE INVENTION

It is one object of the invention to disclose a system for genetic analysis that reads a plurality of input parameters, wherein the input parameters include a file-path to a mutation input file and store the mutation input file, exemplarily in .vcf or .maf format. The mutation input file is comprised of a mutated genetic sequence, and the sorting mutations in the mutation input file are based on the starting position within each chromosome. The computer then receives data identifying chromosome location, start position, reference allele and mutated allele for each mutation presented in the mutation input file and loads the reference genome used to identify the mutations. The system then compares the mutation input file to the input reference genome to generate a new reference genome and a mutation index file, exemplarily in .bed format. The mutation index file provides the location of the wild type and mutated nucleotides in the newly generated reference genome and can be used to quantify allelic expression of aligned next generation sequencing reads which can be used to diagnose a genetic condition.

It is another object of the invention to check the mutation input file for mutations that are duplicated or overlap.

It is yet another object of the invention to check the user's mutation input file by matching non-altered nucleotides to the input reference genome.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is an exemplary embodiment of the hardware of the genomic analysis system;

FIG. 2 is a flowchart detailing an exemplary process by which the software of the present invention operates; and

FIG. 3 is a flowchart detailing another exemplary process by which the software of the present invention operates.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

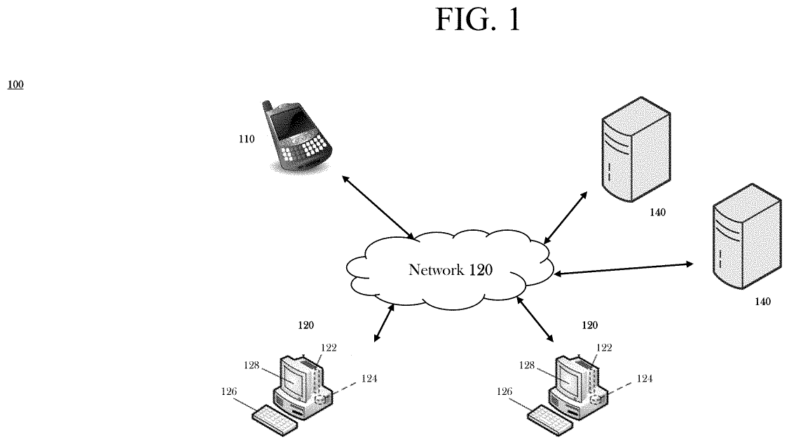

FIG. 1 is an exemplary embodiment of the genetic analysis system. In the exemplary system 100, one or more peripheral devices 110 are connected to one or more computers 120 through a network 130. Examples of peripheral devices 110 include smartphones, servers with databases that contain genetic/genomic data, and any other devices that can be used to collect genetic data that are known in the art. The network 130 may be a wide-area network, like the Internet, or a local area network, like an intranet. Because of the network 130, the physical location of the peripheral devices 110 and the computers 120 has no effect on the functionality of the hardware and software of the invention. Both implementations are described herein, and unless specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices 110 and the computers 120 will both include or be attached to communication equipment. Communications are contemplated as occurring through industry-standard protocols such as HTTP or HTTPS.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable devices (e.g. smartphones, smartwatches, tablets). In one embodiment, each of the peripheral devices 110 and each of the computers 120 of the system may have software related to the system installed on it. In such an embodiment, system data may be stored locally on the networked computers 120 or alternately, on one or more remote servers 140 that are accessible to any of the peripheral devices 110 or the networked computers 120 through a network 130. In alternate embodiments, the software runs as an application on the peripheral devices 110.

In certain embodiments, the software of the present invention is comprised of a script, referred to herein as “MAXX.py” or “MAXX,” which generates a customized reference genome and an accompanying mutation index file based on a user's input of mutations, reference genome, and *.gtf file. Customized reference genomes improve alignment of next generation sequencing reads that contain a predefined indel mutations. This capability to increase alignment sensitivity of predefined mutations can potentially be used to improve detection of circulating cancer cells, improve patient prognosis and enhance personalized therapeutic approaches for cancer patients.

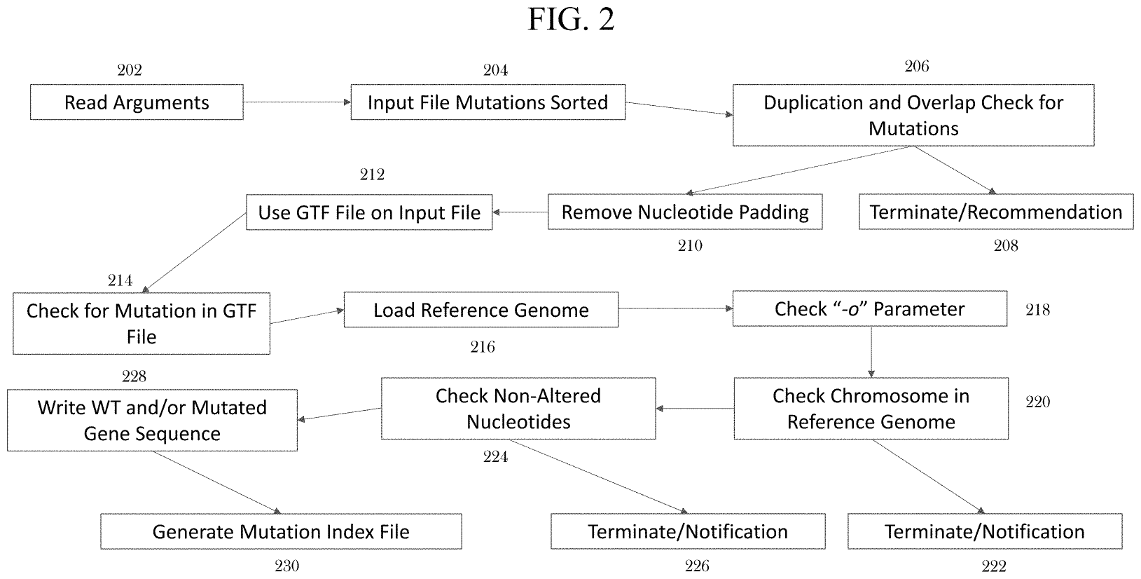

FIG. 2 is a flowchart detailing an exemplary process by which the software of the present invention operates. In general, the software, generates a customized reference genome based on a user's input of mutations, exemplarily in the .vcf or .maf format. To generate a mutated gene sequence, the software alters the wildtype version of the gene to a mutated version. That is achieved by “cutting” the wild type gene where a mutation has been identified by the user and then inserting the altered nucleotides.
gene_sequence=chromosome[(gene_start_position−1): gene_end_position]mutant_sequence=gene_sequence[:(mutation start_position—gene_start_position−1)]+mutation+gene_sequence[(mutation_start_position+len(reference_allele)−gene_start_position−1):]
   The software is thus able to generate a mutated version of a gene that contains multiple mutations. To accurately incorporate multiple mutations into a single gene, the software uses a shifting algorithm to properly alter mutated sequences that have an insertion and/or a deletion in them.
mutant_sequence=gene_sequence
shift=0
for mutation in mutated gene:
   mutant_sequence=mutant_sequence[:(shift+mutation_start_position−gene_start_position−1)]+mutation+mutant_sequence [(shift+mutation_start_position+len(reference_allele)−gene_start_position−1):]
   shift=shift+len(mutant_allele)−len(reference_allele)

At the step “Read Arguments,” 202, the MAXX script will read in arguments from the command line. In certain embodiments, MAXX has a total of 8 input parameters, but only 4 of them are required. Those exemplary required parameters are as follows: (1) -m (required) is the path to the user's mutation input file, preferably in .vcf or .maf format. The mutation input file is the list of mutations that the user wants MAXX to generate a customized reference genome for; (2) -f (required) is the path to the input reference genome that was used to identify mutations from next generation sequencing data; (3) -g (required) is the path to the input GTF file, which identifies the location of genes and transcripts within the input reference genome; (4) -s (required) is the name of the sample/dataset MAXX is being performed on. The output files will be labeled with this input parameter. The optional parameters are: (5) -t DNA, -t gene, or -t transcript lets the user decide the nucleotide format for the mutated allele in the newly generated reference genome. -t DNA extends the mutation in the 3′ and 5′ direction based on the nucleotide padding parameter (-p), -t gene outputs the whole mutated sequence of the gene containing a mutation (default), and -t transcript outputs the whole mutated sequence of the transcript containing a mutation. The (6) -m yes is used if a gene has more than one mutation in it. In that scenario, MAXX will merge the mutations into a single mutated sequence and will output only one version the mutated allele. The -m default creates multiple alleles for mutations that overlap within the specified -t parameter. If -m yes is used on a dataset that has overlapping mutations, MAXX will terminate and the user will be asked to remove the -m yes parameter; (7) -o append or -o genes (optional) lets the user decided if the MAXX output reference genome should only contain the wild type and mutated gene sequence of genes from the mutation input file (default) or if MAXX should append the mutated gene/transcript sequences to the input reference genome (-o append), or generate a reference genome that contains all gene sequences or transcript sequences from the GTF file and the MAXX mutated gene/transcript sequences (-o genes); and (8) -p number (optional) lets the users add a specific amount of nucleotide padding, based on nucleotides from the input reference genome, to the 5′ and 3′ ends of wildtype and mutated nucleotide sequences.

At the step “Input File Mutations Sorted,” 204, mutations in the mutation input file are put into a dictionary and genes with multiple mutations are sorted based on their starting mutation position within each chromosome. This ordering of mutations allows MAXX to easily utilize a shifting algorithm to generate mutated nucleotide sequences that contain two or more mutations. In certain embodiments, this is performed using the Python sorting command. At the step “Duplication and Overlap Check for Mutations,” 206, the software checks if any mutations are duplicated in the mutation file and determines if any mutations overlap with each other. If a mutation is duplicated than the software of the present invention stores only one copy. If mutations overlap with each other and the parameter -m yes is set, then at “Terminate/Recommendation” 208, the software terminates and outputs a recommendation that the user either removes the overlapping mutation or removes the -m yes parameter. At “Remove Nucleotide Padding,” 210, nucleotides that are the same character (A, T, C, G) and overlap at the same positions in the reference allele and mutated allele within the mutation file are removed. At the step “Use GTF File on Input File,” 212, the software uses the GTF file to identify the chromosome location, start position, and end position for each gene that overlaps with a mutation from the mutation input file.

At the step “Check for Mutation in GTF File,” 214, if the -tDNA parameter is not set and mutation does not occur within a gene/transcript in the GTF file, the software removes the mutation from the analysis and outputs a notification to the user.

At the step “Load Reference Genome,” 216, the standardized reference genome is loaded into memory. At the step “Check ‘-o’ Parameter,” 218, if the parameter “-o append” is applied, the mutated alleles will be appended to a copy of the input reference genome. If the parameter “-o genes” is applied, the new reference genome will include all gene sequences from the GTF file and the MAXX mutated gene sequences. Otherwise, only the mutated and wildtype gene sequence of each gene within in the mutation input file will be extracted from the standardized reference genome and written to the new customized reference genome preferably under the “>Gene_Name” tag.

At the step “Check Chromosome in Reference Genome,” 220, the software checks if the chromosome associated with each mutation in the mutation input file is present in the standardized reference genome. If any of the chromosomes do not match up, at “Terminate/Notify GTF” 222, the software terminates and outputs a notification asking the user to find a matching GTF file and standardized reference genome. Otherwise, the software proceeds to the step “Check Non-Altered Nucleotides,” 224.

At the step “Check Non-Altered Nucleotides,” 224, non-altered nucleotides in the mutation input file will be checked against the standardized reference genome to make sure they match. If they do not match, at “Terminate/Notification” 226, the software terminates and outputs a notification asking the user to find a matching GTF file and standardized reference genome.

At the step “Write WT and/or Mutated Gene Sequence,” 228, the mutated sequence for each gene in the mutation input file will be generated and written to the new reference genome preferably under the “>Gene_Name_mut#”. If the -o parameter is set to “default” or “genes,” the wild type sequence is also written to the mutation input file. If the user added the “-m yes” parameter, a shifting algorithm will be used to generate a single mutated sequence for mutations that have overlapping genomic regions. Otherwise, there will be a mutated sequence for each mutation. The shifting algorithm iterates over all mutations within a gene from the smallest gene position to the largest gene position and keeps tract of the difference of nucleotides between the wild type and mutated sequences in order to match the position of the mutation in the wildtype sequence. For example, if a SNP or MNP mutation is present, then no shifting will occur. However, if an insertion mutation is present, then the mutated sequence will shift forward according to the number of inserted nucleotides, and if a deletion mutation is present, then the mutated sequence will shift backwards according to the number of deleted nucleotides.

At the step “Generate Mutation Index File,” 230, the software generates a mutation index file, which identifies where the wild type nucleotides and mutated nucleotides are located in the new customized reference genome. The customized reference genome, which enhances alignment of next generation sequencing reads containing alterations by providing the corresponding sequence, may then be used to diagnose cancers and other conditions that originate due to genetic mutations.

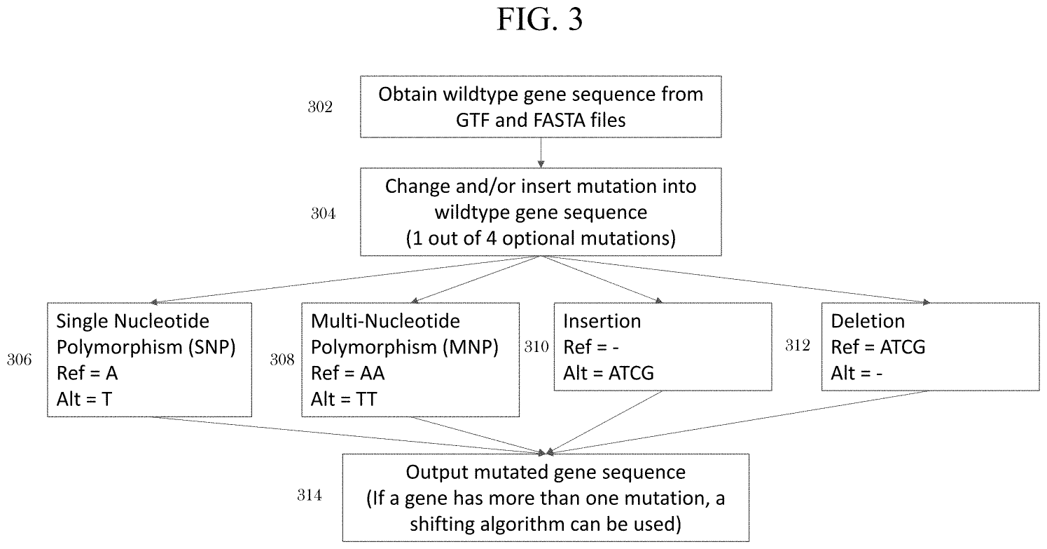

FIG. 3 is a flowchart detailing another exemplary process by which the software of the present invention operates in writing a mutated gene sequence. The process commences with the software obtaining wildtype gene sequences from GTF and FASTA databases 302. The GTF file is used to obtain the nucleotide sequence of a gene or transcript from the FASTA file. Once the wild type sequence is obtained, the software then creates a mutated sequence by changing or inserting one or more mutations into the wildtype nucleotide sequences. The modification of the wildtype sequence is achieved by “cutting” the sequence at the location of the mutations and inserting the new nucleotides. For deletion mutations, an empty string is used to replace the missing nucleotides in the wild type sequence. The software has the option to modify the gene sequence with four types of mutations: a single nucleotide polymorphism 306, a multi-nucleotide polymorphism 308, an insertion 310, and/or a deletion 312. The software then outputs a mutated gene sequence 314, for genes with more than one mutation, the software will either use a shifting algorithm or generate a mutated gene sequence for each individual mutation.

The software of the present invention has numerous applications. The customized gene sequences can be used to improve personalized medicine for cancer patients by identifying genes that are essential to the individual's tumor progression. This is through MAXX's ability to enhance NGS allelic detection by providing the exact sequencing for each known allele. In cancer, the identification of genes that contain a mutation but only express the wild type allele are potentially important for tumor progression due to selective silencing of the mutant allele and may act as therapeutic targets. This molecular phenomenon has been observed in pancreatic cancer.

The customized gene sequences can also be used to enhance personalized immune therapy for cancer patients by improving the ranking of actionable neo-antigens. A major criterion for an actionable neo-antigen is that it needs to be expressed by the tumor. With MAXX generated reference genomes, we can more fully interrogate if a particular genomic alteration is expressed, which will influence if the neo-antigen should be utilized in CAR T-cell therapy. CAR T-cell therapy is currently FDA approved for patients with acute lymphoblastic leukemia, non-Hodgkin lymphoma and multiple myeloma.

In other applications, customized reference genomes can be used to improve the sensitivity of detecting circulating tumor cells and/or cell free tumor DNA/RNA. Tumor DNA/RNA, either within a cell or cell free, is often at very low concentrations within the blood and is unique to individual patients. But with the use of MAXX, we can create personalized reference genomes to enhance detection of tumor DNA/RNA within a blood sample. The ability to detect tumor DNA/RNA within blood samples is an idea way to monitor previously treated cancer patients for cancer recurrence. Using the techniques of the present invention, cancer may be better diagnosed and treated using a pharmaceutically acceptable amount of an anti-cancer drug, as known to those of ordinary skill in the art.

The customized gene sequences of the present invention also reduce the need for exome sequencing when RNA-sequencing data is available. Customized reference genomes can be used to quickly and efficiently integrate RNA-sequencing data for clinically relevant mutations that have previously been discovered. Moreover, the customized gene sequences can also be used to improve validation of CRISPR experiments in next generation sequencing data. Customized reference genome can thus be used to quantify the success rate of a CRISPR experiment that has undergone RNA-sequencing.

The customized reference gene sequences can also be used to improve patient cancer prognosis by better identifying the common evolutionary pathways by which cancers mutate in order to progress their tumor phenotype.

The foregoing description and drawings should be considered as illustrative only of the principles of the invention. All references cited herein are incorporated in their entireties. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All reference cited herein are incorporated by reference in their entirety.

1. A method of genetic analysis, comprising the steps of:
reading a plurality of input parameters, wherein the input parameters comprise a path to a mutation input file;
storing the mutation input file, wherein the mutation input file is comprised of a mutated genetic sequence;
sorting mutations in the mutation input file based on starting position;
receiving data identifying chromosome location, start position, reference allele and mutated allele for each mutation in the mutation input file;
loading a standardized reference genome;
comparing the mutation input file to the standardized reference genome; and
generating a mutation index file, wherein the mutation index file identifies a location of wild type and mutated nucleotides in the customized reference genome, wherein the mutation index file is used to diagnose a genetic condition.

2. The method of claim 1, wherein the mutation index file is used to quantify the number of next generation sequencing reads aligned to the wild type allele or mutant allele, wherein the quantification is performed using allelic expression of mutations.

3. The method of claim 2, wherein the genetic condition is diagnosed using the allelic expression of mutations.

4. The method of claim 1, wherein the genetic condition is a cancer.

5. The method of claim 1, further comprising requesting a new mutation input file if mutations in the mutation input file are duplicated or overlap.

6. The method of claim 1, wherein the comparison of the mutation input file to the standardized reference genome comprises matching non-altered nucleotides in the mutation input file against the standardized reference genome.

7. The method of claim 1, wherein the customized reference genome is comprised of a separate mutated gene sequence for each mutation in the mutation input file or a merged gene sequence that includes all of the mutations from the mutation input file.

8. The method of claim 1, wherein the data identifying chromosome location, start position, and end position for each gene with a mutation from the mutation input file is in a GTF file format.

9. A genetic analysis system, wherein a computer:
reads a plurality of input parameters, wherein the input parameters comprise a path to a mutation input file;
stores the mutation input file, wherein the mutation input file is comprised of a mutated genetic sequence;
sorts mutations in the mutation input file based on starting position;
receives data identifying chromosome location, start position, and end position for each gene with a mutation in the mutation input file;
loads a standardized reference genome;
compares the mutation input file to the standardized reference genome; and
generates a mutation index file, wherein the mutation index file identifies a location of wild type and mutated nucleotides in the customized reference genome, wherein the mutation index file is used to diagnose a genetic condition.

10. The system of claim 9, wherein the mutation index file is used to quantify the number of next generation sequencing reads aligned to the wild type allele or mutant allele, wherein the quantification is performed using allelic expression of mutations.

11. The method of claim 10, wherein the genetic condition is diagnosed using the allelic expression of mutations.

12. The system of claim 9, wherein the computer further requests a new mutation input file if mutations in the mutation input file are duplicated or overlap.

13. The method of claim 9, wherein the genetic condition is a cancer.

14. The system of claim 9, wherein the comparison of the mutation input file to the standardized reference genome comprises matching non-altered nucleotides in the mutation input file against the standardized reference genome.

15. The system of claim 9, wherein the customized reference genome is comprised of a separate mutated gene sequence for each mutation in the mutation input file or a merged gene sequence that includes all of the mutations within a gene from the mutation input file.

16. The system of claim 9, wherein the data identifying chromosome location, start position, and end position for each gene in the mutation input file is in a GTF file format.
